(19) United States
(12) Patent Application Publication
(10) Pub. No.: US 2009/0131537 A1
(43) Pub. Date: May 21, 2009

(54) Wound healing compositions

(76) Inventor: John Jacob Wille, JR., Chesterfield, NJ (US)

Correspondence Address: MILDE & HOFFBERG, LLP, 10 BANK STREET, SUITE 460, WHITE PLAINS, NY, 10606, US

(21) Appl. No.: 12/291,699

(22) Filed: Nov. 13, 2008

Related U.S. Application Data
(60) Provisional application No. 61/003,109, filed on Nov. 14, 2007.

(51) Int. Cl. A61K 31/07; A61P 17/02

(52) U.S. Cl. 514/725

(57) ABSTRACT

Formulations and methods for treating wounds utilizing these formulations are disclosed. The formulations accelerate wound healing by providing a unique serum-free cellular nutrient medium that supports wound healing of mammalian skin in the absence of protein growth factors. The protein-free composition contains physiological levels of a retinoid compound. This retinoid-containing composition enhances epidermal wound healing of both normal acute and chronic wounds by stimulating the growth of the adult epidermal keratinocytes without the need of any protein growth factors. The wound healing active composition may be used in combination with a topical wound gel preparation including both proteinaceous and non-proteinaceous biopolymers and hydrogels.

### FIELD OF THE INVENTION

The present invention relates to new compositions and methods for using such compositions for enhancing skin wound healing and repair of damaged skin. More particularly, the invention relates to wound healing compositions that employ a serum free nutrient medium specialized to grow epidermal keratinocytes of a mammalian animal in combination with at least one retinoid without the need for any protein growth factors.

### BACKGROUND OF INVENTION

The creation of a wound healing and topical skin care formulations that contain multiple ingredients presents many difficulties and challenges due to unanticipated behavior of any particular ingredient in the final formulation. This is particularly the case when it is an amino acid aqueous formulation to be applied topically to intact skin or skin wounds. Typical vehicles that may be used to convey an aqueous skin or wound healing composition include gellen gum (U.S. Pat. No. 7,262,179), hydrocolloids (U.S. Pat. No. 6,201,164), hydrogels U.S. Pat. No. 7,083,806) and any other suitable pharmaceutical or cosmetic vehicle.

Previous first-aide preparations sold over the counter as wound treatments are generally restricted to monographed active ingredients that lack a claim for anything other than improvement of minor cuts and bruises. Among the most widely used compounds in wound dressing is allantoin derived from plants. It has anti-irritant and moisturizing activity that has been claimed in U.S. Pat. No. 4,560,678. Another class of wound care preparations deals almost exclusively with combating bacterial infections. The quaternary compound, trans and cis traumatic acid salts are claimed in U.S. Pat. No. 5,567,716 by Della Valle, et al., (1966) as having cicatrizant activity with bacteriostatic, anti-viral, antifungal and antibiotic activity. Compositions containing disinfectives that have been approved for minor cuts and abrasions are disclosed in U.S. Pat. No. 4,401,651 by Knutson (1983) for the use of povidine iodine a wound disinfective and to improve wound healing. There are many wound healing preparations on the market targeted to disinfecting burn wounds and chronic wounds, and still another category of wound healing preparations pertain to improving the wound environment by removing excess wound exudates through there water absorbing capacity. Further, chronic and burn wounds have been typically treated with a variety of enzymes that aim to remove dead tissue by enzymatic debridement to obtain a clean wound bed prior to application of other standard of care modalities.

Topical preparations containing the flavonoid botanicals, gefarnate and sofalcone, for treatment of wounds have been claimed in U.S. Pat. No. 5,457,128 by Yanagawa (1995). Recently, Haskell (2007) in U.S. Pat. No. 7,175,860 has disclosed a composition derived from nut husks that enhance wound healing. Herbal remedies also abound for treatment of wounds. They include preparations containing Royal Jelly and other bee honeys. In general, these act to prevent bacterial colonization of wounds but lack the ability to treat clinical wound infections through their reduction of water activity and hyper-tonicity. The herb, tumeric, which has been shown to have excellent anti-inflammatory activity, has been claimed as a wound healing agent in U.S. Pat. No. 5,401,504.

Recent patent literature has disclosed wound care preparations containing amino acids, vitamins, antioxidants, botanical extracts or particular compounds isolated from plants. Klein in WO/20000/069403 teaches a topical skin dermatological composition that contains two dicarboxylic amino acids, aspartic acid and glutamic acid. In U.S. Pat. No. 6,048,843 a topical composition is disclosed containing amino acid in combination with either interferon of thymidine derivatives for treating viral or inflammatory diseases. In U.S. Pat. No. 5,425,954 a topical amino acid-vitamin complex composition is disclosed for both pharmaceutical and cosmetic uses. The mixture contains the vitamins, panthenol and alpha-tocopherol acetate, and arginine, isoleucine, leucine, methionine phenylalanine threonine, and valine amino acids. The vitamin nicotinamide has been disclosed for topical application to promote angiogenesis, reepithelization and wound healing in U.S. Pat. No. 4,725,609, and panthenolate salts are claimed as topical medicaments in U.S. Pat. No. 4,602,036. A combination of vitamins, fatty acids and antioxidants are disclosed as wound healing agents in U.S. Pat. No. 5,652,274. Ascorbic acid (vitamin C) in combination with collagen type I and Vitamin E acetate is disclosed by Obi-Talbot in U.S. Pat. Nos. 6,046,160 and 6,187,743 to heal wounds in a dressing formulation. Peshoff in U.S. Pat. No. 7,094,431 disclosed fat-soluble vitamins in combination with a calcium channel blocker and zinc oxide. Finally, Lewis et al., (1992) in U.S. Pat. No. 5,156,847 disclose the free acid of taspine, an aporphinoid, as a wound healing agent when dissolved in dimethylsulfoxide. Later, Winter et al., disclosed in U.S. Pat. No. 5,932,617 taspine derivatives with better solubility profiles as a wound healing composition. Lipid compounds have been claimed in several patents. The use of phospholipid vesicles to deliver ATP is claimed as a wound healing composition in U.S. Pat. No. 7,041,312 and in U.S. Pat. No. 5,714,478 Spiegel discloses the use of a sphingo-phosphosphorylcholine as a wound healing agent. Leibovich (2001) discloses a therapeutic wound healing composition containing an effective amount of an inhibitor of mono-adenosine disphosphate-ribosyl transferase as a therapeutic dermatological is claimed in U.S. Pat. No. 6,187,822. A wound healing composition containing deoxyribonucleosides is disclosed in U.S. Pat. No. 5,770,582.

Advanced wound healing also embraces the use of growth factor proteins and cytokines as actives that make claims for stimulating wound healing of both acute and chronic wounds. Fahey et al., (1992) in U.S. Pat. No. 5,145,676 claim that cytokines or mixture thereof of cytokines that are capable of binding heparin when administered locally promote wound healing. Among the cytokines mentioned are the inflammatory cytokines MIP-1, MIP-1alpha, MIP-1beta, and MIP-2. The protein growth factor, beta-transforming growth factor, TGF-beta, is disclosed as a wound healing agent in U.S. Pat. No. 5,981,606. Similarly, purified growth factors involved in fibroblast and keratinocyte growth have been disclosed in numerous patents. In particular, Lynch and Antoniades (1991) in U.S. Pat. No. 4,983,581 claimed the use of insulin-like growth factor, IGF-1, in combination with TGF-beta as a wound healing composition. The same inventors have disclosed other growth factor pairs as wound healing composition. For example in U.S. Pat. No. 4,874,746 (1989) they disclose platelet-derived growth factor, PDGF, in combination with transforming growth factor, TGF-alpha, while in U.S. Pat. No. 5,019,559 (1991) they disclose the combination PDGF and insulin-like growth factor-2, IGF-II, and finally in U.S. Pat. No. 5,034,375 they disclose the growth factor combination PDGF and epidermal growth factor, EGF, as useful in the process of wound healing. The angiogenic growth factor, human basic fibroblast growth factor, is disclosed by Moscatelli et al., (1991) in U.S. Pat. No. 4,994,559 as useful in pharmaceutical preparations for wound healing. Also, the granulocyte-macrophage colony stimulating growth factor, GM-CSF, has been disclosed by Pierce et al., (2004) as useful in promoting accelerated wound healing in mammals in U.S. Pat. No. 6,689,351. Klagsbrun et al., (2001) discloses in U.S. Pat. No. 6,235,884 heparin binding mitogens including an epidermal growth factor homologous segment (HB-EHM) that stimulate fibroblast, epithelial cells and antibodies that recognize, and purified nucleic acids that encode these growth factors as well as isolated polypeptides, vectors that contain such nucleic acid, and cells that harbor such vectors. These growth factor preparations are claimed to accelerate the rate of wound healing. The protein, beta-transforming growth factor, TGF-beta, is disclosed as a wound healing agent in U.S. Pat. No. 5,981,606. Recently, the use of leptin and agents that modulate leptin and leptin receptors has been disclosed in U.S. Pat. No. 7,261,881 as possible wound healing agents.

The use and delivery of platelet lysates as wound healing agents is disclosed in U.S. Pat. No. 5,156,938 by Knighton (1992). And a platelet gel wound healant containing activated growth factors, ascorbic acid and antioxidant vitamins A and E are disclosed in U.S. Pat. No. 7,112,342 by Worden (2006). A gel formulation containing platelet-derived growth factors or purified platelet-derived growth factor (PDGF), useful for topical wound healing are claimed in U.S. Pat. No. 5,457,093 by Cini et al., (1995) which later became FDA approved wound healing preparation for the treatment of chronic wounds marketed by Ethicon, Inc (Sommerville, N.J.). Lindenbaum in U.S. Pat. No. 5,461,030 relates to formulations and methods for treating wounds, which comprise the use of effective amounts of a serum free cellular nutrient medium in combination with an effective amount of human growth hormone. It describes the use of a serum-free culture medium for the growth of normal human epidermal keratinocytes that is supplemented with nanogram per ml amount of human growth hormone.

A polypeptide inhibitor of matrix metalloproteinases is disclosed in U.S. Pat. No. 7,060,795, which Quirk claims to be useful in treating chronic wounds. Chemical analogs of the cardiac drugs, angiotensin II an angiotensin II fragments, are claimed by Rodgers et al., in U.S. Pat. No. 7,022,675 to be useful in accelerating wound healing in a mammal. The latter invention is primarily directed at healing the dermis and its vascularization. The enzyme placental alkaline phosphatase in combination with serum growth factors is disclosed in U.S. Pat. No. 7,011,965 as an agent that stimulates fibroblast proliferation and wound healing. A wound healing composition that contain an effective amount of activated growth factors in combination with ascorbic acid, at least one retinoid, and at least one antibiotic is claimed in U.S. Pat. No. 6,303,112. A composition and method for healing wounds is disclosed in U.S. Pat. No. 6,541,447 that uses 5% by weight of ovalbumin in combination with 1% phenoxyethanol 0.5% carbomer, and 0.3% triethanolamine in the form of a cream, powder, lotion, gel, emulsion or ointment. A wound healing composition, containing alpha-1-antitrypsin for the treatment of chronic wounds is disclosed in U.S. Pat. No. 6,638,909. A method for promoting wound healing that uses the proteins tropoeleastin and lysyl oxidase to bind with and cross-link wound tissues to increase its tensile strength is claimed in U.S. Pat. No. 6,808,707.

Growth factors and other protein pharmaceutical fall under guidelines of the U.S. Food and Drug Administration as biologics and or pharmaceuticals, which engenders serious concerns about their effects on the human body in excess of the amount made by the body. Moreover, none of the growth factors and growth factor products or treatment modalities specifically stimulates wound healing by promoting the epidermal cell growth alone. To overcome the latter limitation, biological dressing composed entirely of epidermal keratinocytes and formed in culture as three-dimensional epithelial sheet have been used for wound healing as skin grafts (Wille, Jr., U.S. Pat. No. 5,292,655, 1994; Wille. Jr., U.S. Pat. No. 5,686,307: Wille, Jr., U.S. Pat. No. 5,834,312; Wille, Jr., U.S. Pat. No. 5,912,175; Wille, Jr., U.S. Pat. No. 6,162,643; Wille, Jr., U.S. Pat. No. 7,037,721).

Previously, applicants have disclosed a serum free tissue culture medium that supports the growth of epidermal keratinocyte cells in U.S. Pat. No. 5,292,655. It also discloses by way of included patent references many of the previous techniques claimed as wound healing biological dressing, all of which that employed serum-containing medium to growth keratinocytes. A method for the formation of a histologically-complete skin substitute is disclosed in U.S. Pat. No. 5,686,307; it describes a composition of a serum-free medium that supports the growth of epidermal keratinocytes and the formation of a complete living skin substitute. An autologous epidermal skin graft formed in culture using a novel serum-free basal medium is described in U.S. Pat. No. 5,686,307. The methods and media formulations disclosed in these patents were subsequently used in a clinical trial to successfully heal venous stasis ulcers (Wille et al., 2007). These patents all employ protein growth factors to growth the skin grafts. In U.S. Pat. No. 7,037,721, Wille discloses a novel serum free medium that grows a three-dimensional skin graft in culture without the need for protein growth factors. The novel medium composition replaces the protein growth factors with retinyl acetate, a vitamin A fatty acid.

In the past, vitamin A and its derivatives (retinoids) have been examined for their wound healing effects. It is well known that nutrition plays an important part in wound healing. In this regard, Rojas and Philips (1999) reported that patients with chronic leg ulcers showed diminished levels of Vitamin A. Earlier, Hung et al., (1989) reported that topical retinoid treatment of surgically-produced wounds on porcine skin accelerated wound healing, while prolonged treatment actually retarded wound healing. As early as 1966 Boss et al., reported that Vitamin A induced both autologous and homograft skin rejection. Although no explanation of this delay was proposed, presumably wound healing was retarded due to hypervitaminosis A, which heightens immunity in patients treated orally with high doses of vitamin. Recent reports (Popp et al., 1995; Paquette et al, 2001) demonstrate that all-trans retinoic acid (tretinoin) stimulates increase in granulation tissue in photoaged skin and chronic wounds. Moreover, a positive effect of vitamin A on reepithelialization of wounds was been reported by Klein (1999), who found that vitamin A stimulated reepithelialzation of standardized surgical wound in epidermis of patients put on a 1% vitamin A acid diet. In addition, Popp et al., (1995) reported that tretinoin dramatically accelerated wound healing of photodamaged skin. Varani et al., (1990) reported that retinoic acid stimulated human dermal fibroblast proliferation, which occurred in a serum-free medium when the calcium ion concentration was elevated to 1.4 mmol/L. Sustained proliferation of serum free cultures of adult and newborn foreskin keratinocytes was achieved in F12 medium supplemented with EGF, insulin, transferrin, hydrocortisone, cholera toxin, endothelial cell growth factor and 1×10.sup.−7M retinoic acid when cells were seeded on collagen coated culture dishes (Jee et al, 1990). Duell et al (1997) reported that topical application of retinal, retinaldehyde, and retinyl palmitates do not produce erythema unlike retinoic acid, and resulted in increased epidermal thickness. Topical retinoic acid therapy was reported to heal sun-exposed and sun-protected adult skin equally well whereas normal neonatal skin was less responsive (Varani et al., 1998). Retinoids have also been shown to improve wound healing compromised by steroid therapy (Anstead, 1998; Wicke et al., 2000). These authors concluded that steroid and retinoids have antagonistic effects on growth factors and collagen deposition during wound healing. Earlier, Varani et al. (1989) disclosed that all-trans retinoic acid stimulates the growth of adult human keratinocytes cultured in growth factor-deficient medium. Early passage keratinocytes were incubated for 1 or 2 days in serum free keratinocyte growth medium (MCDB 153) supplemented with EGF, insulin and 1.4 mM Ca.sup2+ or in growth factor derived keratinocyte basal MCDB 153 medium. The cells were concomitantly treated with all-trans retinoic acid (0.1-2.5 ng/ml). Treatment with all-trans retinoic acid inhibited proliferation of keratinocytes that were rapidly growing in the growth factor-supplemented medium. By contrast, all-trans retinoic acid treatment of keratinocytes in growth factor deficient medium, in which the cells were growth arrested, stimulated growth. Stimulation was observed in a serum free medium lacking not only protein growth factors, but also the hormones, hydrocortisone, ethanolamine, and phosphoethanolamine. The rate of keratinocyte proliferation in the retinoid-stimulated cultures was approximately 35% of the maximal proliferation rate observed in growth factor supplemented medium. It should be noted that the optimal concentration of all-trans-retinoic acid required to produce the effect was 0.5 ng/ml (1.6×10.sup−6M). This is about 100-fold greater than the physiological concentration, and is present in amount known to be damaging to cell membranes. Lower concentrations of all-trans retinoic acid were ineffective. In addition, Marcelo and Dunman (1997) reported that retinoic acid stimulates the growth of essential fatty acid-supplemented keratinocytes. These results were observed in keratinocyte cultures grown in a serum free medium (MCDB 153) that was supplemented with the protein growth factors, EGF, Insulin, and bovine pituitary extract (BPE) using a protein free defined medium called PF86-a (Rikimaru et al, 1990) with 85% serum free medium MCDB 153 (U.S. Pat. No. 4,673,649). No explanation or hypothesis was given as to what elements(s) of the composition were responsible for the ability of this medium to support keratinocyte growth in the absence of protein growth factors.

The use of a retinoid to support sustained growth of keratinocytes, as disclosed in U.S. Pat. No. 7,037,721, suggests that this novel serum-free media could be used as a wound healing composition.

### OBJECTS OF THE INVENTION

Thus, by replacing protein growth factor requirements with a retinoid, the issue of using pharmacological proteins as wound healing agents is eliminated. This was the starting point for the development of a novel and unobvious topical wound healing composition.

The present invention eliminates the use of growth factors in a defined medium resulting in many technical and commercial benefits. In order to accomplish this goal, the present invention proposes replacing EGF and IGF-1 with a novel amino acid composition in combination with an effective amount of retinyl acetate. With this composition, sustained growth of human keratinocytes is readily achieved. In addition, retinyl acetate stimulates keratinocytes proliferation at physiological concentration unlike the reported effect of all-trans retinoic acid (Varani et al., 1989).

It is therefore a principal object of the present invention to provide novel wound healing formulations.

It is another object of the present invention to provide a basal medium that maintains the viability of the intact skin as an organ.

It is still another object of the present invention to provide a composition that accelerates wound healing by maintaining a moist wound healing environment, that supports rehydration of the damaged skin with stratum corneum barrier defects.

It is yet another object of the present invention to provide novel compositions that accelerates wound healing of chronic wounds with the provision of an acceptable topical skin delivery gel.

### BRIEF DESCRIPTION OF THE INVENTION

The present invention relates to formulations and methods for treating wounds utilizing these formulations. The formulations accelerate wound healing by providing a unique serum-free cellular nutrient medium that supports wound healing of mammalian skin in the absence of protein growth factors. The protein-free composition contains physiological levels of a retinoid compound. This retinoid-containing composition enhances epidermal wound healing of both normal acute and chronic wounds by stimulating the growth of the adult epidermal keratinocytes without the need of any protein growth factors. Another aspect of the invention is the use of the wound healing active composition in combination with a topical wound gel preparation including both proteinaceous and non-proteinaceous biopolymers and hydrogels.

In the preferred embodiments according to the present invention the formulations include an effective amount of a retinoid that stimulates the autocrine production of growth factors that are resident in the epidermal margins of the wound. The most preferred embodiment of the retinoid that accelerates wound repair is an effective amount of retinyl acetate present in the novel serum-free nutrient medium in physiological amounts, having a concentration in the range from 1×10⁻⁹ M to 5×10⁻⁷ M.

### BRIEF DESCRIPTION OF THE DRAWINGS

FIG. 1: Photograph of culture dishes showing the effect of different combinations of growth factors on the clonal growth of HaCat keratinocytes. Std, standard serum free; Ins, Insulin; EGF, epidermal growth factor; RetAc, retinyl acetate. Magnification is 1.2×.

FIG. 2: Photograph of culture dishes showing the effect of retinol acetate on the induction of autocrine growth of HaCat keratinocytes. Control, serum free medium supplemented with EGF (E) and insulin (I), D0, day 0; D1, day 1; Ins, 5 ug/ml insulin; RAct, retinal acetate, 3×10.sup.−8M. Magnification is 1.2×.

FIG. 3: Photomicrograph showing result of indirect immunofluorescent staining pattern of normal human foreskin keratinocytes cultured on glass cover slips in serum free medium supplemented with EGF and insulin and stained with c-neu antibodies A) when glass slips were prepared in the absence of pretreatment with alkaline phosphatase enzyme or B) when slipes were prepared with alkaline phosphatase pretreatment. Arrows point to fluorescent staining. Total magnification is 1750×.

FIG. 4: Photomicrograph showing result of indirect immunofluorescent staining pattern of normal human foreskin keratinocytes cultured on glass slips in standard medium containing insulin and retinoic acid and stained with c-neu antibodies (A) when slips were prepared without alkaline phosphatase pretreatment, or (B) when slips were prepared with alkaline phosphatase pretreatment. Arrows point to fluorescent staining. Total magnification=700×; B, total magnification is 1750×.

FIG. 5: Photograph of culture dishes showing the effect of a specific receptor tyrosine kinase inhibitor on HaCat keratinocytes cultured in (A) standard medium (Std) on day 1 (D1) in (B) autocrine growth medium (Ins+RAc, D1), and (C) autocrine growth media in the presence of receptor tyrosine kinase inhibitor (RTKI). Magnification is 1.2×.

FIG. 6: Photograph of culture dishes showing the effect of varying concentrations of retinyl acetate on wound healing of HaCat keratinocytes after a midline wounding in the wound healing zone (WHZ) and complete healing after 24 hours of autocrine growth medium in both 3E-8 (3×10.sup.−8M), 3E-7 (3×10.sup−7M) and incomplete healing in 3E-6 (3×10.sup−6M) and 3E-5 (3 10.sup.−5M) retinyl acetate. Control cells were cultured in serum-free medium supplemented with EGF and insulin. Magnification is 1.2×.

FIG. 7: Photomicrograph of HaCat keratinocytes showing (A) complete wound healing in the wound healing zone (WHZ) in the presence of 3×10.sup.−7M retinyl acetate, and (B) no healing in the WHZ in the presence of 3×10.sup.−5M retinyl acetate. Total magnification is 3500×.

### BRIEF DESCRIPTION OF THE PREFERRED EMBODIMENTS

In describing the present invention in the specification a number of terms will be used. The term “wound” is used throughout the specification to describe skin wounds, which are treated with the formulations and methods of the present invention. Skin wounds include several classes of skin damage including: punctures, incisions, excisions, lacerations, abrasions and burns. More particularly, the object of the present invention is the use the wound healing composition to accelerate the healing of chronic wounds including, pressure sore, diabetic and venous stasis ulcers.

The term “delivery polymer” throughout the specification is used to describe both biopolymers including but not exclusively in collagen, gelatin, natural polysaccharides, chitosan-type hydrogels, and synthetic polymers including but not exclusively sodium carboxymethyl cellulose, and hydrogels such as hydroxyethylmethacrylate (HEMA), glycerolmethacrylate (GMA), and polyvinylpyrrolidone (PVP) and polyethylene glycol (PEG).

The term “serum-free medium” is used throughout the specification to describe a medium, which contains no serum, and in combination with the retinoid of choice that comprises the wound healing composition of the present invention. The basal nutrient medium, given as “Formulation A” of the present invention, comprises the following elements: (a) essential amino acid, (b) non-essential amino acids, and (c) vitamins selected from the group consisting of biotin, folate, lipoate, niacinamide, pantothenate, pyroxidine, riboflavin thiamine, and cobalamine (B12). All of these elements are present in a number of basal nutrient media including MCDB 153, MCDB 154 and HECK 110. The optimal concentrations of these media elements are described in U.S. Pat. No. 7,037,721, which also discloses their use to promote the growth of normal human epidermal keratinocytes. A topical wound healing composition is given in “Formulation B.” It eliminates most amino acids, all vitamins and trace elements and glucose and is supplemented with retinyl acetate in the range of 1×10.sup.−9M to 5×10.sup.10−7M.

Other serum free nutrient medium that support the growth of normal human keratinocytes that may be used according to the present invention may include commercially available media including Ham's F12, MCDB 153, and MCDB 154.

The term “autocrine-stimulating active” is used throughout the specification to describe the action of a retinoid not limited to retinyl acetate, in promoting wound healing in the absence of protein growth factors including insulin, epidermal growth factor (EGF), and bovine pituitary extract (BPE). The preferred embodiment is retinyl acetate. It is a naturally occurring fatty acid ester form of Vitamin A that binds to and activates retinoid receptors, and induces cell differentiation. It belongs to the retinoid family of isoprenoid compounds. They have been shown to be essential in maintaining the viability of mammalian epithelial tissues (Wolf, 2000). Absence of Vitamin A in the diet of pregnant mice results in loss of vision in the offspring, and to squamous cell metaplasia of tracheal epithelium in hamsters (Wille & Chopra, 1988). Moreover, retinoids have chemopreventive effects on mouse skin tumorigenesis model (Wille, 2003). Earlier studies (Wille, 1986) showed that retinoic acid inhibits the clonal growth of normal human keratinocytes when added to a serum free growth factor supplemented medium.

In studies presented in Example 1 we show that growth factor-deficient serum free media supplemented with both retinyl acetate and insulin are sufficient to support the growth of the HaCat cell line of immortalized human keratinocytes, whereas serum free media supplanted with insulin only is less effective. Further, we show that this ability is due to the autocrine production of another member of the EGF family of growth factors, heparin-binding EGF protein (Klagsbrun et al., 2001).

In Example 2 we show that the phenomenon of retinoid-induced autocrine growth of keratinocytes is dependent on growth factor receptor-mediated protein tyrosine kinase stimulation that regulates cell cycle progression through the mitogen-activated protein kinase (MAPK) pathway. In Example 3 we disclose two formulations of a serum free media that enhance wound healing of chronic wounds. Example 4 we show the effect of the wound healing composition of Example 4 on the healing of epidermal wounds in an in vitro epidermal keratinocyte “epithelial sheet” skin model.

### DETAILED DESCRIPTION OF THE PREFERRED EMBODIMENTS

The preferred embodiments of the present invention will now be discussed.

### Example 1

### Effect of Growth Factor-Retinoid Interactions on HaCat Cell Proliferation

Normal human keratinocytes can only grow in a serum free defined nutrient medium such as MCDB 153, MCDB 154 or HECK 109 supplemented with hormones (hydrocortisone, ethanolamine and phosphoethanolamine) if it has also been supplemented by at least two protein growth factors, insulin (Ins⁺) and epidermal growth factor (E⁺).

In the following experiment, the effect of on keratinocyte growth of adding single or multiple additives to standard medium (Std) was examined in a clonal growth assay performed according to the methods described previously (Wille et al., 1984). Briefly, a sterile 60 mm² plastic disposable Petri dish is seed with 5,000 high cloning HaCat cells, and refed complete (E⁺I⁺) serum free HECK 109 medium at incubated at 37° C. for 24 to 48 hours. The cells are washed with ice-cold Standard HECK 109 medium and refed various combinations of growth factors and retinyl acetate (RetAC).

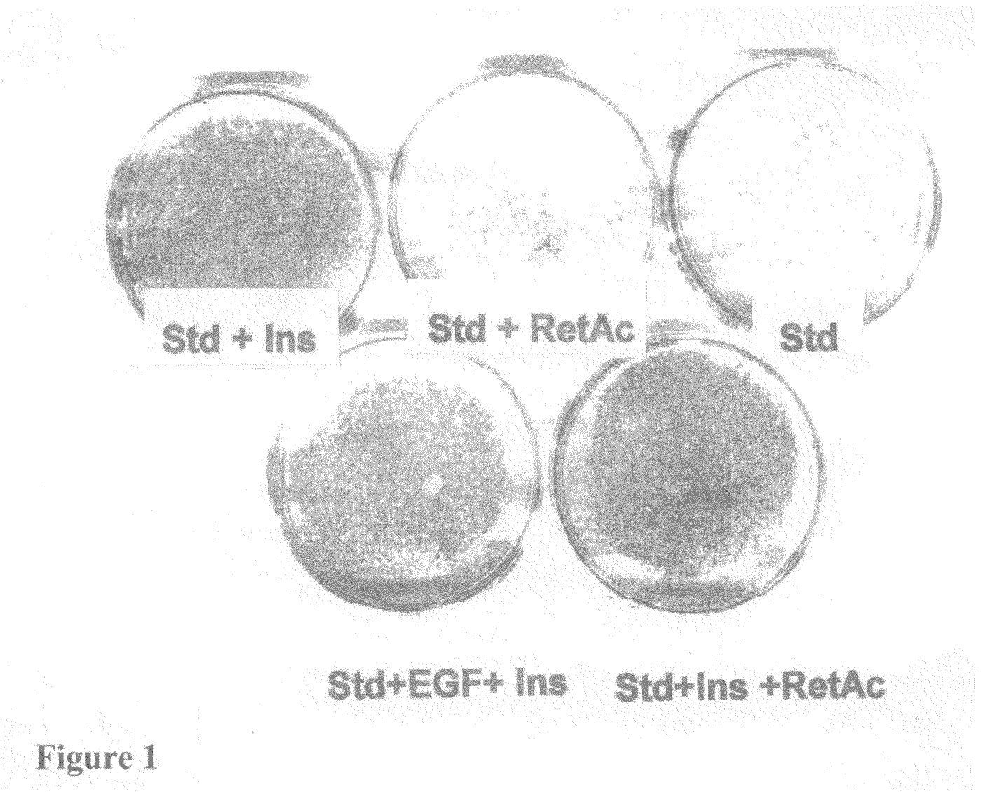

FIG. 1 presents a photograph showing the results of incubating the HaCat seeded clonal assay dishes various supplements. The cells were fixed with 50% ethanol and stained with 0.2% crystal violet stain. This imparts a blue color to the cells. The stained dishes were then photographed. It can been seen that little if any keratinocyte growth occurred either in just standard medium (Std) or in standard medium supplemented with 3×10.sup.−8 M (Std+RetAc). By contrast, HaCat cells in the clonal growth dishes containing standard media supplemented with insulin (5 ug/ml) only (Std+Ins) or with 5 ng/ml EGF and 6 ug/ml insulin (Std+EGF+Ins) were stimulated to grow. By far, the best result was stimulation of keratinocyte growth when standard medium was supplemented with the combination: 5 ug/ml insulin and 3×10.sup−8M retinyl acetate.

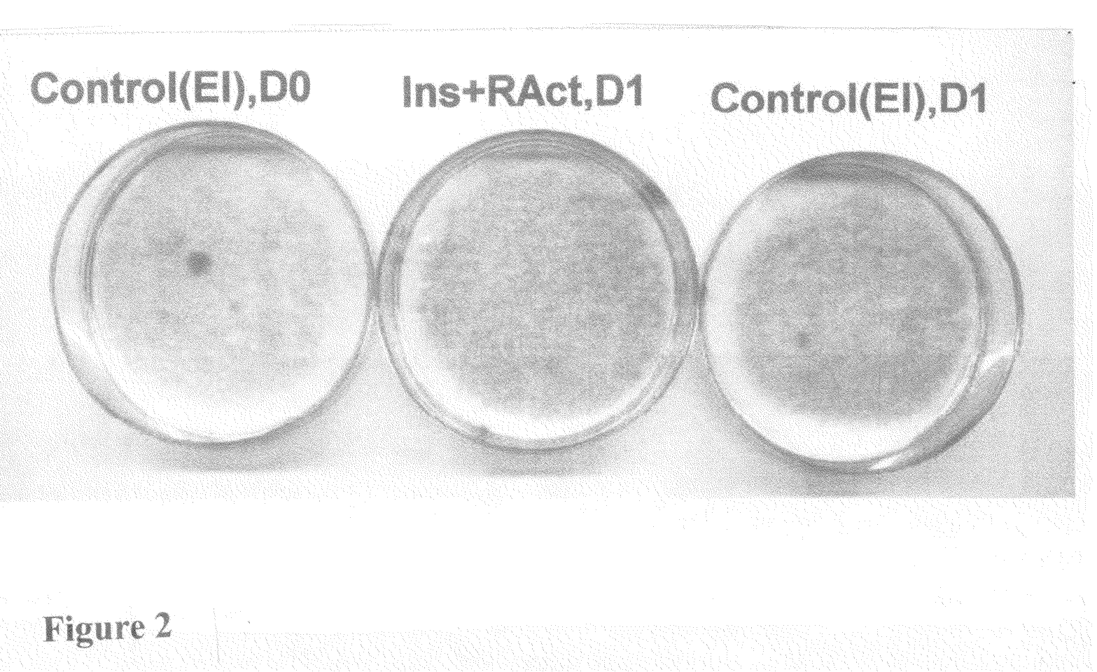

In a second series of experiments the HaCat keratinocyte clonal growth after one day (D1) was compared for the combination: insulin plus retinyl acetate (Ins+Rac, D1) and the combination: EGF plus insulin (EI, D1). FIG. 2 presents the results in a photograph showing that the combination (I+Rac, D1): 5 ug/ml insulin (I), plus 3×10.sup.−8M retinyl acetate (Rac), stimulated keratinocyte growth as well as the combination (EI): 5 ug/ml insulin, 5 ng/ml EGF, and bovine fetal serum (I+E+F). These results demonstrate that retinyl acetate can replace EGF as a necessary prerequisite for HaCat keratinocyte growth. In these studies, insulin was also a necessary growth factor when used in combination with retinyl acetate. This may reflect the often-observed phenomenon of “carry-over” of bound EGF on cell surface growth factor receptor. However, in additional tests anti-EGF antibodies were added to the standard medium combination of insulin plus retinyl acetate, which did not eliminate the effectiveness of the insulin plus retinyl acetate combination in stimulating keratinocyte growth.

### Example 2

### Retinoid-Induced Autocrine Growth of Keratinocytes is Dependent on Phosphorylation of Growth Factor Receptor-Mediated Activation of the MAPK Pathway

The growth of HaCat keratinocytes in standard nutrient medium without EGF raises a question as to how the retinoid, retinyl acetate, achieves this. Reference has been made to the finding that retinoid treatment might induce autocrine production of a member of the EGF family. Earlier studies by Shipley et al., demonstrated that normal human keratinocytes secrete their own EGF-like growth factor. In this instance, a heparin-binding member of the EGF family of growth factors, hb-EGF, which they called amphiregulin. Amphiregulin is normally secreted by keratinocytes in sufficient amounts only when the cell density of the culture exceeds 2×10.sup.4 cells per cm².

### Experiment 1

### Detection of erbB Antibody

In order to determine whether retinoids like retinyl acetate induced autocrine secretion of an EGF-like protein growth factor that binds to an EGF-like cell surface receptor, we designed an experiment using the erbB antibody purchased from Oncogene Sciences (Manhasset, N.Y.) and performed indirect immunofluoresnce (IIF) cytochemistry on fixed normal human neonatal foreskin keratinocytes prepared according to previous studies (Wille et al., 1984). The technique of indirect immunofluorescene was performed according to the antibody manufacturers instructions for c-neu antibody. The neu oncogene was discovered after serial transformation of cells using rat neuroblastoma DNA. The isolated oncogene hybridizes with v-erbB and has 50% homology to the normal EGF receptor, a tyrosine kinase type enzyme protein. The proto-oncogene for neu is designated c-erb-2.

These facts suggest that c-neu antibody should react with keratinocyte cells possessing an EGF-receptor that responds to retinoid-stimulation of autocrine growth.

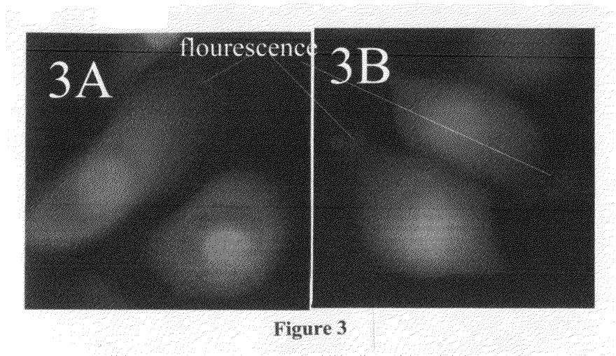

Preliminary studies showed that c-neu antibody reacted positively with cells in early G1 phase of the cell cycle, and with post-mitotic suprabasal keratinocytes committed to terminal differentiaton. FIG. 3A shows that staining was dispersed over the entire cytoplasm with a tendency toward a monopolar distribution of finely and particulate stained foci. FIG. 3B shows that cells treated with alkaline phosphatase, an enzyme that removes phosphate groups from proteins, before straining with c-neu antibody had a uniquely localized distribution of positive stain which was no longer generalized to cytoplasm but to highly restricted to focal adherence plaques where one cell makes contact with another cells. This indicates that phosphorylation of c-neu receptors redistributes the c-neu type receptors away from areas of cell-to-cell contact in keratinocytes growing in the absence of retinoid treatment We interpret this to mean that phosphorylation of c-neu favors cell separation associated with keratinocytes proliferation.

### Experiment 2

### Effect of Retinoid Treatment on c-neu Expression in Proliferating Keratinocyte Cultures

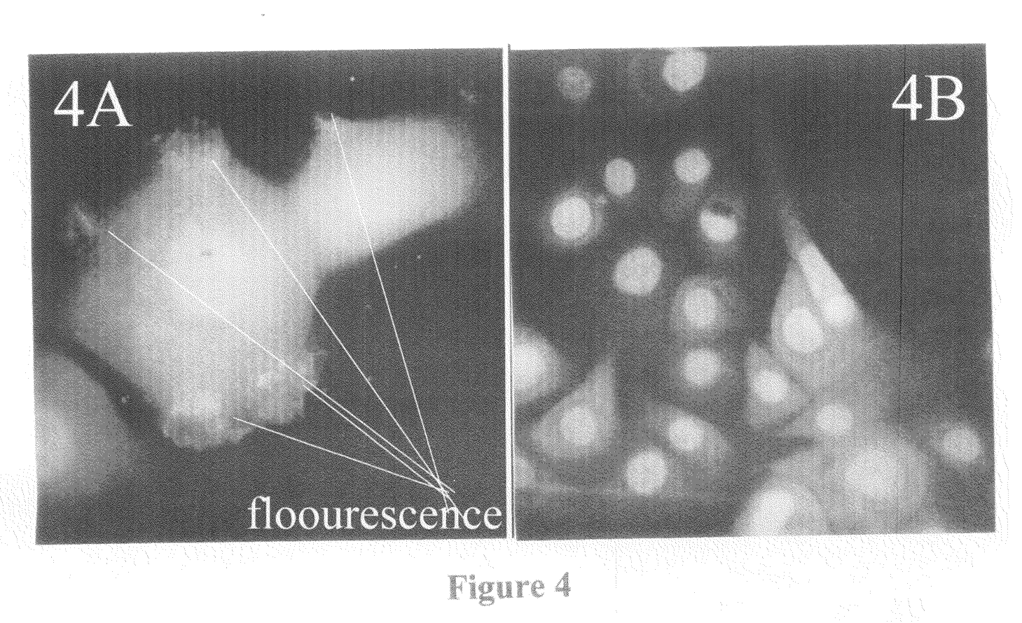

Normal human keratinocytes were propagated in a serum free medium composition containing 5×10.up.−8M all-trans retinoic acid, fixed and prepared for c-neu staining and detection by IIF as described in Experiment 1. FIG. 4A shows results of retinoic acid in the absence of any alkaline phosphatase pre-treatment. The staining pattern revealed that virtually all of the positive staining was strictly localized to focal adherence areas of the cell where cell to cell contacts predominant. FIG. 4B shows that retinoic acid induced a labile phosphorylation of c-neu only at the focal adherence sites. We interpret this to mean that retinoid stimulation of autocrine growth proceeds through phosphorylation of an EGF-like receptor at points of cell-to-cell contact, which usually is only seen is post-mitotic suprabasal cells. These results demonstrate that retinoid-induced autocrine growth of keratinocytes is dependent on the expression of a new retinoid-inducible EGF-like receptor in post-mitotic suprabasal cells, which are then able to reenter the cell cycle when stimulated by autocrine secreted hbEGF. These results are in full agreement with an earlier report (Xiao et al., 1999) identifying a heparin binding EGF-like growth factor as the target in the intercellular regulation of epidermal basal cell growth by suprabasal retinoic acid receptors.

### Experiment 3

### Inhibition of Retinoid-Induced Autocrine Growth by an Inhibitor of Growth Factor Receptor Phosphorylation

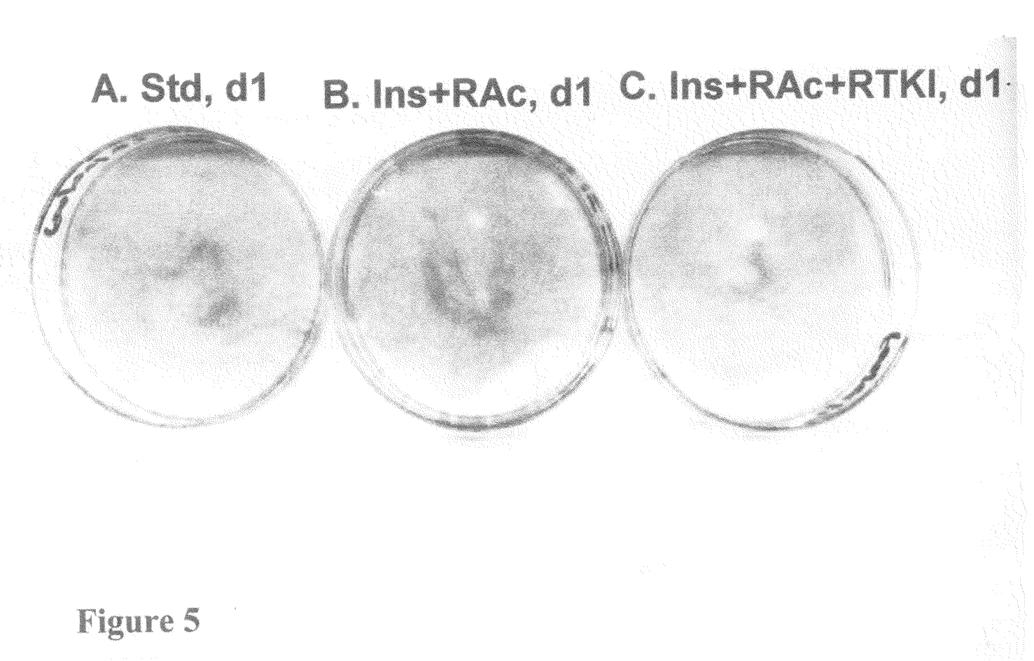

In this experiment a selective inhibitor of receptor tyrosine protein phosphokinase was added to a culture of HaCat keratinocytes immediately prior to replacing the growth factor replete medium with HECK 110 serum free medium containing the combination insulin and retinyl acetate to induce autocrine growth. FIG. 5 presents a photograph showing three culture dishes with the following growth conditions: A. Standard growth medium (Std, d1); B. Standard growth medium containing the combination: 5 ug/ml insulin plus 3×10.sup.−8M retinyl acetate (Ins+RAc, d1) and C. standard medium containing the combination: 5 ug/ml insulin, 3×10.sup.−8M retinyl acetate and 2 uM of receptor tyrosine protein kinase inhibitor (Ins+Rac+RTKI, d1).

The results show that there was no additional growth in dish C over that seen in dish A. whereas there was significant growth in B well above that for either A or C dishes.

We interpret this result as demonstrating that retinoid-induced autocrine growth of HaCat keratinocytes is dependent on phosphorylation of the tyrosine residue in the newly induced EGF-like growth factor receptor protein and that failure to phosphorylate this residue impedes further signal transduction required for phosphorylation events in the MAPK pathway that initiates gene transcription necessary for cell cycling.

### Example 3

### Wound Healing Formulations

For the purposes of specifying the composition of topical formulations that embody the present invention two related formulations (A, and B) are given in the Tables 1 and 2.

TABLE 1
Formulation A: Wound Healing Composition for Autocrine
Growth of Epidermal Keratinocytes.

| | | Concentration in final medium | |
|---|---|---|---|
| Stock | Component | mg/l | mol/l* |
| 1 | Arginine•HCl | 210.7 | 1.00 × 10⁻³ |
| | Histidine•HCl•H₂0 | 33.54 | 1.60 × 10⁻⁴ |
| | Isoleucine allo-free | 6.6 | 4.50 × 10⁻⁵ |
| | Leucine | 66.0 | 0.50 × 10⁻³ |
| | Lysine•HCl | 18.3 | 1.00 × 10⁻⁴ |
| | Methionine | 8.95 | 6.00 × 10⁻⁵ |
| | Phenylalanine | 16.67 | 1.00 × 10⁻⁴ |
| | Threonine | 23.8 | 2.00 × 10⁻⁴ |
| | Tryptophan | 10.2 | 0.50 × 10⁻⁴ |
| | Tyrosine | 5.40 | 3.00 × 10⁻⁵ |
| | Valine | 35.13 | 3.00 × 10⁻⁴ |
| | Choline | 13.96 | 1.00 × 10⁻⁴ |
| | Serine | 63.06 | 6.00 × 10⁻⁴ |
| 2 | Biotin | 0.0146 | 6.00 × 10⁻⁸ |
| | Calcium Pantothenate | 0.285 | 1.00 × 10⁻⁶ |
| | Niacinamide | 0.03663 | 3.00 × 10⁻⁷ |
| | Pyridoxal•HCl | 0.06171 | 3.00 × 10⁻⁷ |
| | Thiamine•HCl | 0.3373 | 1.00 × 10⁻⁶ |
| | Potassium chloride | 111.83 | 1.50 × 10⁻³ |
| 3 | Folic acid | 0.79 | 1.80 × 10⁻⁶ |
| | Na₂HPO₄•7H₂0 | 536.2 | 2.00 × 10⁻³ |
| 4a | Calcium chloride•2H₂0 | 14.7 | 1.00 × 10⁻⁴ |
| 4b | Magnesium chloride•6H₂0 | 122.0 | 6.00 × 10⁻⁴ |
| 4c | Ferrous sulfate•7H₂0 | 1.39 | 5.00 × 10⁻⁶ |
| 5 | Phenol red | 1.242 | 3.30 × 10⁻⁶ |
| 6a | Glutamine | 877.2 | 6.00 × 10⁻³ |
| 6b | Sodium pyruvate | 55.0 | 5.00 × 10⁻⁴ |
| 6c | Riboflavin | 0.03764 | 1.00 × 10⁻⁷ |
| 7 | Cysteine•HCl | 37.6 | 2.40 × 10⁻⁴ |
| 8 | Asparagine | 13.2 | 1.00 × 10⁻⁴ |
| | Proline | 34.53 | 3.00 × 10⁻⁴ |
| | Putrescine | 0.1611 | 1.00 × 10⁻⁶ |
| | Vitamin B₁₂ | 0.407 | 3.00 × 10⁻⁷ |
| 9 | Alanine | 8.91 | 1.00 × 10⁻⁴ |
| | Aspartic acid | 3.99 | 3.00 × 10⁻⁵ |
| | Glutamic acid | 14.71 | 1.00 × 10⁻⁴ |
| | Glycine | 7.51 | 1.00 × 10⁻⁴ |
| 10 | Adenine | 12.16 | 9.00 × 10⁻⁵ |
| | Inositol | 18.02 | 1.00 × 10⁻⁴ |
| | Lipoic acid | 0.2063 | 1.00 × 10⁻⁶ |
| | Thymidine | 0.7266 | 3.00 × 10⁻⁶ |

stocks (cont'd)
Trace

| | | | |
|---|---|---|---|
| element | Copper sulfate•5H₂0 | 0.00025 | 1.00 × 10⁻⁹ |
| T | Selenic acid | 0.00387 | 3.00 × 10⁻⁸ |
| | Manganese sulfate•5H₂0 | 0.00015 | 1.00 × 10⁻⁹ |
| | Sodium silicate•9H₂0 | 0.1421 | 5.00 × 10⁻⁷ |
| | Ammonium molybdate•4H₂0 | 0.00124 | 1.00 × 10⁻⁹ |
| | Ammonium vanadate | 0.00059 | 5.00 × 10⁻⁹ |
| | Nickel chloride•6H₂0 | 0.00012 | 5.00 × 10⁻¹⁰ |
| | Stannous chloride•2H₂0 | 0.000113 | 5.00 × 10⁻¹⁰ |
| | Zinc chloride•7H₂0 | 0.1438 | 5.00 × 10⁻⁷ |
| Solids S | Glucose | 1081.0 | 6.00 × 10⁻³ |
| | Sodium acetate•3H₂0 | 500.0 | 3.70 × 10⁻³ |
| | Sodium bicarbonate | 1176.0 | 1.40 × 10⁻² |
| | Sodium chloride | 7022.0 | 1.20 × 10⁻² |
| | HEPES | 5240.0 | 2.20 × 10⁻² |

Actives

| | | | |
|---|---|---|---|
| A | Retinyl acetate | 0.01 | 3 × 10⁻⁸ |
| | Ethanolamine | 6.1 | 1 × 10⁻⁴ |
| | Phosphoethanolamine | 14.11 | 1 × 10⁻⁴ |
| | Hydrocortisone | 0.0363 | 5 × 10⁻⁷ |

The above Formulation A is the full list of components necessary to grow keratinocytes in a serum free culture under autocrine growth control. This formulation is very complex and has been simplified for use in a topical wound healing gel as shown in Table 2 below.

### Example 4

### Topical Skin and Wound Healing Formulation

With the elimination of protein growth factors specified as necessary for the serum free growth of normal human keratinocyte in culture and their replacement by retinyl acetate, it was necessary to re-examine the minimal basal nutrient requirements for stimulating epithelial cell growth in wounds. Amino acid in topical preparation are valuable because of their chemical nature (amino and carboxylic groups). They and are also known as “zwitter ions,” which can stabilize the skin's acid mantle and perform a protective function. The amino acid, arginine is know to have skin-moisturizing and belong to agents known as natural moisturizing factors, NMF (Wille, 2006). For this and other reasons, Formulation B includes 0.2% arginine.

The final composition of Formulation B was achieved by assessing the criticality of each component in a new topical wound composition. In particular, they are the essential components required for autocrine stimulation of basal keratinocytes by wound edge healing from a sheet of contiguous keratinocytes. The following additional changes were made to Formulation B. Most importantly is the inclusion of retinyl acetate. It is the key to autocrine growth of keratinocytes. Formulation B also dispenses with most of the amino acids of Formulation A as they are believed to be available in sufficient amounts in blood plasma and not critical to stimulate keratinocyte proliferation from the wound edge. The critical amino acids retained are the seven amino acid listed above in Table 2: arginine, isoleucine-allo free, methionine, phenylalanine, threonine, tryptophan and tyrosine at the concentration shown in Table 2. All trace elements, vitamins and glucose were also eliminated as these too are present in sufficient amounts in blood plasma. There was no need to have phenol red in the composition as a pH indicator as the composition was adjusted to pH 7.2 by the provided salts. The tonicity of the medium Formulation B is isotonic due to the combined concentrations of the added salts. Hepes (N-(2-OH-ethyl-piperazine-N′-(2-ethane sulfonic acid) an organic buffer in Formulation A was added as long-term stabilization of pH in the gel formulation. The final formulation components are shown below as Formulation B.

TABLE 2
Formulation B: Topical Skin and Wound Healing Composition

Concentration in
final medium

| | Mg/L | Moles/L |
|---|---|---|
| Amino acids | | |
| Arginine | 2107 | 1.00 × 10⁻² |
| Isoleucine allo-free | 6.6 | 0.50 × 10⁻⁴ |
| Methionine | 8.95 | 6.00 × 10⁻⁵ |
| Phenylalanine | 16.67 | 1.00 × 10⁻⁴ |
| Threonine | 23.8 | 2.00 × 10⁻⁴ |
| Tryptophan | 10.2 | 0.50 × 10⁻⁴ |
| Tyrosine | 5.40 | 3.00 × 10⁻⁵ |
| Autocrine stimulating agent | | |
| Retinyl acetate | 1.00 | 3.0 × 10⁻⁶ |
| Solid Salts | | |
| Sodium pyruvate | 55.0 | 5.00 × 10⁻⁴ |
| Sodium acetate•3H₂0 | 500.0 | 3.70 × 10⁻³ |
| Sodium bicarbonate | 1176.0 | 1.40 × 10⁻² |
| Sodium chloride | 7022.0 | 1.20 × 10⁻² |
| Potassium chloride | 111.83 | 1.50 × 10⁻³ |
| Na₂HPO₄•7H₂0 | 536.2 | 2.00 × 10⁻³ |
| Calcium chloride•2H₂0 | 14.7 | 1.00 × 10⁻⁴ |
| Magnesium chloride•6H₂0 | 122.0 | 6.00 × 10⁻⁴ |
| Ferrous sulfate•7H₂0 | 1.39 | 5.00 × 10⁻⁶ |
| Hepes (N-(2-OH-ethylpiperazine | | |
| N′-(2-ethane sulfonic acid | 5423 | 2.20 × 10⁻² |

### Example 5

### Effect of Retinyl Acetate on Healing of Wounded Epidermal Sheets

For the purpose of demonstrating a wound healing effect of retinyl acetate under autocrine growth conditions, HaCat keratinocyte cell line was employed. Keratinocytes were grown in a serum free medium supplemented with 5 ng/ml EGF and 5 ug/ml insulin and 0.2% fetal bovine serum and reseeded at 1×10.sup.5 cells per cm² in to 35 mm 2 circular disposable Petri dishes and incubated at 37° C. until the cells completely filled the dish. These confluent cultures were then washed with ice-cold basal MCDB serum free medium and refed a serum free medium of the present invention whose composition is as shown in Table 1. A 2.5 mm linear wound was made across the midline of the diameter of the confluent culture with a 2.5 mm diameter heat-sealed tip of a 9 inch sterile Pasteur pipette. This resulted in a visible linear wound designated here as a wound healing zone, WHZ. Wound of this diameter were routinely and reproducibly made by this technique and allow study of the effect of any agent that might have an effect on wound healing by measuring the delay in time it takes to fill in the wound gap of the WHZ.

### Experiment 1

### Effect of Retinyl Acetate on WHZ Wound Gap Closure

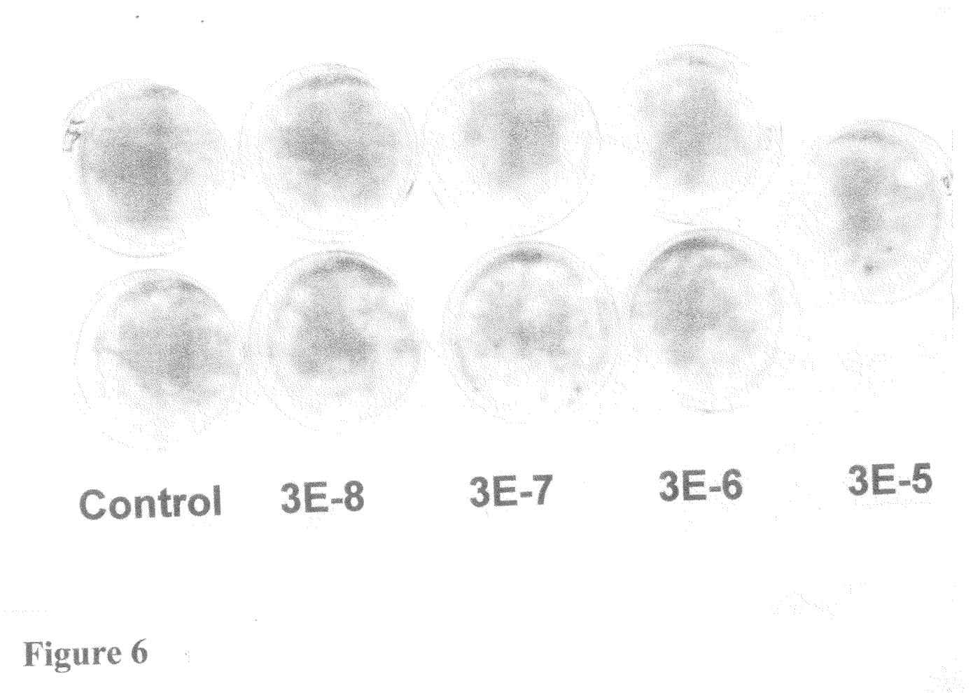

FIG. 6 is a photograph of 9 culture dishes fixed with 50% ethanol and stained with 0.2% crystal violet 24 hours after forming the WHZ and incubated in the serum free medium of this invention containing varying doses of retinyl acetate. The experiment was performed in duplicate dishes. There is only one dish labeled 3E-5 as its duplicate was contaminated with mold and was eliminated. Control dishes were refed serum free medium supplemented with insulin but no retinyl acetate. All of the other dishes were r3efed serum free medium with insulin and increasing amount of retinyl acetate. The mi9d line WHZ was filled by epidermal keratinocyte migration and cell growth in all of the dishes except for cultures that had retinyl acetate at concentrations greater than 3×10.sup.−6 M.

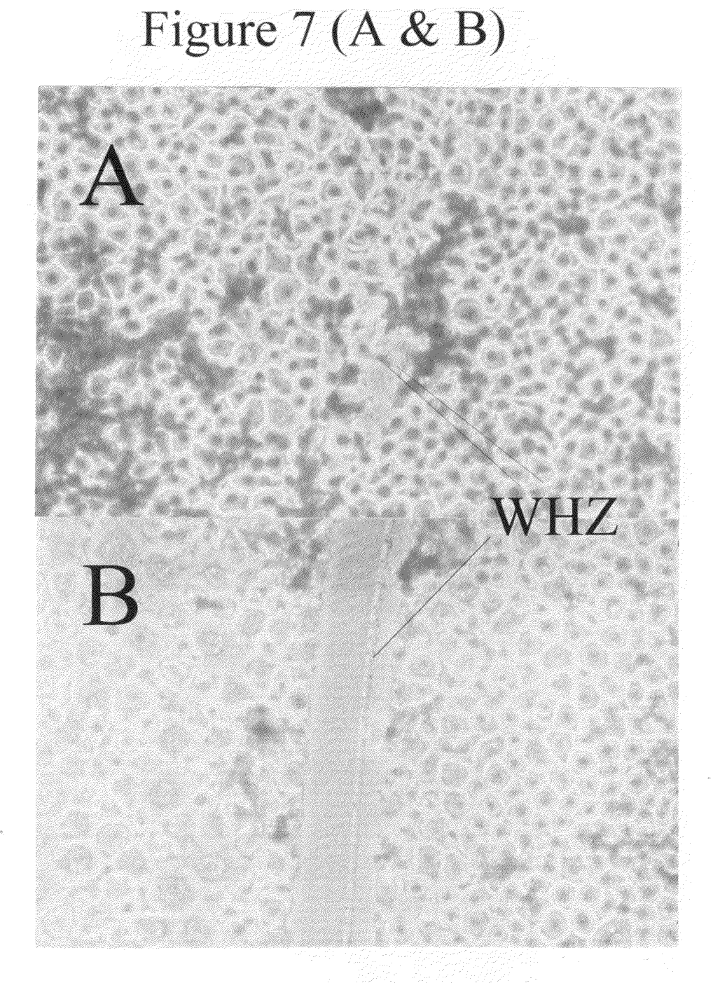

FIG. 7 show photomicrographs comparing wound closure in the WHZ for cultures of HaCat keratinocytes fixed and stained with crystal violet stain and photographed with bright field illumination. FIG. 7A shows complete failure to fill in the WHZ gap at 3×10.sup.−5M compared with virtually 100% healing in the WHZ at 3×10.sup.−7M concentration.

### Example 6

### Aqueous Gel Delivery for Wound Healing Compositions

To deliver a topical wound healing composition to skin wounds a suitable vehicle gel formulation is necessary. Gelatin is often employed as a vehicle to delivery aqueous formulations to skin and has been incorporated in to many wound dressings, e.g., ConvaTec's Stomahesive® ostomy patches. Gelatin is subject to enzymatic dissolution in wound beds due to gelatinases present in wound exudates (Chen et al) as a consequence degraded gelatin would release amino acid and peptides in to the wound milieu with perhaps unintended either positive and or negative consequences for epidermal keratinocyte growth. A fibrin glue mixture has also been advocated for wound healing, simulating a wound clot. Again, this is subject to protease digestion and release of amino acids and peptides. For these reasons we have chosen non-protein gel systems for delivery of Formulation B wound healing composition. Table 3 lists gels that can be employed as compatible delivery vehicles for the wound healing composition of Formulation B.

TABLE3
Aqueous Gel Delivery Systems Compatible for Water-Soluble
Wound Healing Compositions (Formulations A and B).

| Gel Component (%) | Formulation A | Formulation B |
|---|---|---|
| Gelatin | + | + |
| Collagen | + | + |
| Fibrin/fibrinogen | + | + |
| Clay minerals | + | + |
| Carbomer (polyacrylic acid) | + | + |
| Preneutralized polyacrylic acid | + | + |
| Cocamide DEA | + | + |
| Cocamide MEA | + | + |
| Sodium Carboxymethylcellulose | + | + |
| Hydroxyethyl cellulose | + | + |
| Hydroxymethyl cellulose | + | + |
| Hydroxypropylcellulose | + | + |
| Calcium alginate | + | + |
| Corn starch | + | + |
| Locust bean gum | + | + |
| Gum Acacia | + | + |
| Gum Arabic | + | + |
| Gum Guar | + | + |
| Nonionic polyol | + | + |
| Potassium carbomer | + | + |
| Potassium stearate | + | + |
| Gum Xanthan | + | + |
| Chitosan | + | + |
| Anionic acrylic copolymer | + | + |
| Hydroxyethylmethacrylate (HEMA) | + | + |
| Methcrylates copolymer | + | + |
| Methacryloyl ethyl betaine | + | + |
| Glycerolmethacrylate (GMA) | + | + |
| Polyvinylpyrrolidone (PVP) | + | + |
| Polyethylene glycol (PEG) | + | + |

### Example 7

### Liposome Delivery of Wound Healing Compositions (Formulations A and B)

In contrast to the bulk delivery of water soluble components of the present invention, liposome vesicles that incorporated water-soluble components in lipid vesicle is a way of achieving more long-lasting delivery of the wound healing composition. Incorporation of the water-soluble components of Formations A and B can be achieved with the following liposome delivery systems.

TABLE 4
Liposome Encapsulation of Wound Healing Compositions
(Formulations A and B)

| Liposome systems: | Formulation A | Formulation B |
|---|---|---|
| Stabilized natural | + | + |
| lecithin (PC) mixtures | | |
| Synthetic identical | + | + |
| chain phospholipids | | |
| Glycolipid-containing | + | + |
| Liposomes | | |
| Bipolar fatty acids | + | + |
| Methyl/methylene cross-linked | + | + |
| Lipoprotein coated | + | + |
| Carbohydrate coated | + | + |
| Multiple encapsulated | + | + |
| Emulsion compatible | + | + |
| Solid lipid nanoparticles (SLN) | + | + |
| Multiphase polyol-in-oil | + | + |
| (PO) emulsion | | |

There has, thus been shown and described novel retinoid-containing wound healing compositions in the preferred embodiments of the present invention. It is to be understood, that the examples of treatment protocols, cell culture manipulations, choice of retinoids employed in the above examples, and the specific wound healing composition gel delivery vehicle are not limited to those alone but can be any choice of physiologically-acceptable retinoids in combination with any growth factor free serum-free media compositions, and any suitable gel delivery vehicle that are generally useful and employed in wound healing applications and to those familiar with the state of the art in wound healing. Many changes and modifications, variations and other uses and applications of the subject invention will, however, become apparent to those skilled in the art after considering the specification and the accompanying compositions and formulations, which disclose the preferred embodiments thereof. All such changes, modifications, variations and other uses and applications which do not depart from the spirit and scope of the invention are deemed covered by the invention, which is not to be limited only by the claims which follow.

What is claimed is:

1. A wound healing composition for topical application to an external wound of a mammal, said composition comprising:
(1) a serum-free nutrient medium capable of supporting the clonal growth at least one epidermal keratinocyte; and
(2) at least one retinoid compound,
whereby other protein growth factors are unnecessary.

2. The wound healing composition according to claim 1, wherein the retinoid compound is retinyl acetate.

3. The wound healing composition according to claim 1, wherein the retinoid compound has a physiological level having a concentration in the range from 1×10⁻⁹ M to 5×10⁻⁶ M.

4. The wound healing composition according to claim 3, wherein the retinoid compound is retinyl acetate.

5. The wound healing composition according to claim 4, wherein the retinoid acetate has a concentration of approximately 3.0×10⁻⁸ M.

6. An aqueous wound healing composition for topical application to an external wound of a mammal, said composition comprising:
(1) at least one retinoid compound; and
(2) a composition comprising essential and non-essential amino acids, vitamins, mineral salts, lipid metabolites, hydrocortisone, putrescence, adenine, thymidine, glucose, sodium chloride, sodium acetate, organic and inorganic buffers.

7. The aqueous wound healing composition according to claim 6, wherein the retinoid compound is retinyl acetate.

8. The aqueous wound healing composition according to claim 7, wherein the retinyl acetate has a concentration of approximately 3×10⁻⁸ M.

9. The aqueous wound healing composition according to claim 6, wherein the lipid metabolites are selected from the group consisting of lipoic acid, choline, ethanolamine and phosphoethanolamine.

10. The aqueous wound healing composition according to claim 9, wherein all of the lipid metabolites in the group are selected.

11. An aqueous wound healing composition comprising, in combination:
(1) retinyl acetate;
(2) an amino acid selected from the group consisting of: Arginine, Isoleucine allo-free, Methionine, Phenylalanine, Threonine, Trytophane and Tyrosine; and
3) a solid salt selected from the group consisting of sodium pyruvate, sodium acetate, Sodium bicarbonate, Sodium Chloride, Potassium Chloride, Sodium phosphate heptahydrate, dibasic, Calcium Chloride dihydrate, Magnesium Chloride hexahydrate, Ferrous Sulfate heptahydrate and, Hepes (N-(2-OH-ethylpiperazine-N′ (2-ethane sulfonic acid).

12. The aqueous wound healing composition according to claim 11, wherein all of the amino acids in the group are selected.

13. The aqueous wound healing composition according to claim 11, wherein all of the solid salts in the group are selected.

14. The aqueous wound healing composition according to claim 12, wherein all of the solid salts in the group are selected.

15. A wound healing gel formulated as an aqueous solution of the wound healing composition according to claim 6 and comprising one or more thickening agents.

16. A wound healing gel formulated as an aqueous solution of the wound healing composition according to claim 11 and comprising one or more thickening agents.

17. A method for accelerating the healing process in an external wound of a mammal, comprising the step of topically applying the wound healing composition of claim 6 to the wound.

18. A method for accelerating the healing process in an external wound of a mammal, comprising the step of topically applying the wound healing composition of claim 11 to the wound.

19. A method for accelerating the healing process in an external wound of a mammal, comprising the step of topically applying the wound healing gel of claim 15 to the wound.

20. A method for accelerating the healing process in an external wound of a mammal, comprising the step of topically applying the wound healing gel of claim 16 to the wound.
